(19) United States
(12) Patent Application Publication
(10) Pub. No.: US 2003/0170313 A1
(43) Pub. Date: Sep. 11, 2003

(54) Micro-particulate and nano-particulate polymeric delivery system

(76) Inventor: Ales Prokop, Nashville, TN (US)

Correspondence Address: Benjamin Aaron Adler, ADLER & ASSOCIATES, Candle Lane, Houston, TX, 77071, US

(21) Appl. No.: 10/356,139

(22) Filed: Jan. 31, 2003

Related U.S. Application Data
(63) Continuation-in-part of application No. 09/169,459, filed on Oct. 9, 1998.
(60) Provisional application No. 60/062,943, filed on Oct. 9, 1997.

(51) Int. Cl. A61K 9/14; A61K 9/16; A61K 9/50; B01J 13/02; B01J 13/04

(52) U.S. Cl. 424/490; 264/4.1

(57) ABSTRACT

The present invention provides a method of making particles, e.g., nanoparticles that are stable in a physiological environment for at least a day. The nanoparticles comprise polyanionic polymers and polycations in a complex useful for drug delivery. The method comprises the step of capturing droplets comprising the polyanionic polymers in a solution comprising the polycations; or, alternatively, capturing droplets comprising the polycations in a solution comprising the polyanionic polymers. Also provided are methods of use for the particles.

### CROSS-REFERENCE TO RELATED APPLICATION

This application is a continuation-in-part of non-provisional application U.S. Ser. No. 09/169,459, filed Oct. 9, 1998, which claims benefit of provisional U.S. Ser. No. 60/062,943, filed Oct. 9, 1997, now abandoned.

### BACKGROUND OF THE INVENTION

1. Field of the Invention

The present invention relates generally to the fields of pharmaceutical sciences, protein chemistry, polymer chemistry, colloid chemistry, immunology, and biomedical engineering. More specifically, the present invention relates to a novel microparticulate and nanoparticulate system for drug, protein, antigen, or nucleic acid delivery.

2. Description of the Related Art

Microparticulate systems are solid particles having a diameter of 1-2,000 μm (2 mm) and more preferably 1-10 μm (microparticles). Nanoparticulate systems are submicroscopic colloidal particles (nanoparticles) having a diameter of preferably 50-500 nm. Both microparticles and nanoparticles can be formed from a variety of materials, including synthetic polymers and biopolymers such a s proteins and polysaccharides. Both microparticles and nanoparticles are used as carriers for drugs and other biotechnology products, e.g., drugs, proteins, antigens, genes, and antisense oligonucleotides.

In the controlled drug and antigen delivery area microparticles and nanoparticles are formed in a mixture with molecules to be encapsulated within the particles for subsequent sustained release. A number of different techniques are routinely used to make these particles from synthetic or natural polymers, including phase separation, precipitation, solvent evaporation, emulsification, and spray drying, or a combination thereof (1-5).

Microparticles and nanoparticles can be prepared either from preformed polymers, such as polylactic acid, polylactic-glycolic acid (6) or from a monomer during its polymerization, as is the case with polyalkylcyanoacrylates (7). Both of the above technologies have limited application because of the use of organic solvents during their preparation which leave residual organic solvents in the final product. Although the polyalkylcyanoacrylate nanoparticulate technology is also available as a water-based technology (8), animal studies demonstrated a presence of toxic degradation products (9).

Cell encapsulation is a related technology which has been also explored for the purpose of making microparticles and nanoparticles (10). Such particles can be formed either by polymer precipitation, following the addition of a non-solvent or by gelling, following the addition of a large amount of small inorganic ion, e.g., salt, or salting-out and of a complexing polymer of an opposite charge. If enough time is allowed, the particle interior or core can be completely gelled. Usually, the inner core material is typically of a polyanionic nature having a negatively charged polymer, the particle membrane or corona is made from a combination of polycation, such as a positively charged polymer, and polyanion. The core material is usually atomized or nebulized into small droplets and collected in a receiving bath containing a polycationic polymer solution. The reverse situation is also possible in which case the core material is polycationic and the receiving bath is polyanionic.

Several binary polymeric encapsulation systems have been described. An undesirable side effect of these encapsulation systems is that the membrane parameters are tied by a single chemical complex resulting from the ionic interactions. The inability to adjust particle parameters independently has limited the success of this system.

Furthermore, literature data reported on polyelectrolyte complexes indicate that such particles aggregate or dissolve in presence of small amounts of salts and sera. It is well known that the stability of polyelectrolyte complexes, such as binary complexes composed of two polyclectrolytes, in salt solutions is very low (11). The presence of salts, either in the reaction mixture or as an addition to the already formed complex, weakens the electrostatic interactions and favors exchange interactions which lead to a dissociation of the complex. This is particularly pronounced with small molecular salts.

The addition of a low-molecular weight electrolyte, e.g., sodium chloride, potassium chloride, magnesium chloride, is an effective way to destroy polyelectrolyte complexes. It leads to a complete liberation of the polycation from the complex. Similarly, a n addition of small molecular weight anions destroys the complex. All these phenomena occur in presence of serum, a fluid containing a plurality of different electrolytes and hydrophilic substances. In some instances some complexes precipitously aggregate at very low salt concentrations (12-15). In addition to the electrolytic instability in the presence of salt, examination of the physical stability of nanoparticles in the presence of serum is a key problem for in vivo applications. Hydrogel nanoparticles precipitously aggregate and increase in size in the presence of even small amounts of animal sera. Severe aggregation of nanoparticles diminishes their gene transfer potential (16).

To overcome these limitations, a new multicomponent, such as ternary or quaternary, polymeric particle was designed which allows for independent modification of mechanical strength and permeability. Over one thousand combinations of polyanions and polycations were examined as polymer candidates suitable for encapsulation of living cells. Thirty-five combinations were found to be usable. However, the composition and concentrations do not allow for the generation of small microparticles and nanoparticles that are suitable for delivery of a drug, protein, antigen or a nucleic acid. Additionally, microparticles and nanoparticles can be designed that are stable in the presence of salt and sera.

The prior art is deficient in the lack of effective means of drug, protein and antigen delivery, as well as plasmid and oligonucleotide delivery. The present invention fulfills this longstanding need and desire in the art.

### SUMMARY OF THE INVENTION

In one embodiment of the present invention there is provided a method of making a nanoparticle that is stable in a physiological environment for at least a day where the nanoparticle comprises polyanionic polymers and polycations in a complex useful for drug delivery. The method comprises the step of capturing droplets comprising the polyanionic polymers in a solution that comprises the polycations, or, alternatively, capturing droplets comprising the polycations in a solution that comprises the polyanionic polymers such that the complex structure forming the nanoparticle made by this method increases the stability of the nanoparticle in a physiological environment.

In another embodiment of this invention there is provided a method of increasing the entrapment efficiency of a nanoparticle by capturing droplets comprising polyanionic polymers and a salt in a solution comprising polycations, said polyanionic polymers and said polycations sufficient in number to form a ternary or quaternary polymeric nanoparticle structure and forming the nanoparticle structure comprising a polyanionic/salt core and a polycationic/polyanionic complex corona with an excess positive charge on the nanoparticle periphery where the structure of the nanoparticle increases the stability thereof in a physiological environment and thus increasing entrapment efficiency.

In yet another embodiment of this invention there is provided a nanoparticle comprising a core which itself comprises polyanionic polymers and a salt and a corona surrounding the core which comprises a polycationic/polyanionic complex with an excess positive charge on the nanoparticle periphery such that the polyanionic polymers and said polycations are sufficient in number to form a ternary or quaternary polymeric nanoparticle structure.

In yet another embodiment there is provided a method of processing reactor content to remove unwanted residual reactants comprising the steps of sedimenting or centrifuging the reactor content; collecting microparticles or the nanoparticles described herein generated in a stirred reactor; rinsing the particles in an excess of water, buffer or cryopreservation solution; separating the suspension by the sedimentation or centrifugation step; repeating the rinsing and separation steps, and reducing the volume of the suspension to about 1/100th of the initial volume. Also provided are methods of stabiling and cryopreserving the particles.

In still another embodiment of the present invention there is provided a method of adjustment of the biodegradability of polymeric mixtures by contacting an enzyme to a polysaccharide and degrading the polysaccharide at physiological conditions in vivo to degradation products.

In still yet another embodiment of the present invention there is provided a method of introducing an adjuvant to potentiate an immunogenic effect by administering the adjuvant as part of a droplet-forming polymeric mixture. The adjuvant may be an aluminum salt enabling to gel certain polysaccharides. Additionally, the adjuvant may be CMC, CS and HV alginate as droplet forming anionic polymers and aluminum sulfate, calcium chloride and a polycationic polymers as a corona forming mixture.

Other and further aspects, features, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions of the invention briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows the size distribution of nanoparticles with an integrated OVA immunogen.

FIG. 2 shows the charge distribution of nanoparticles with an integrated OVA immunogen.

FIG. 3 depicts the response to subcutaneous nanoparticulate OVA antigen (second column) in terms of anti-OVA antibodies. The bars represent the average+/−SD.

FIG. 4 depicts the response to subcutaneous nanoparticulate TT antigen (second column) in terms of anti-TT antibodies. The bars represent the average+/−SD.

FIG. 5 depicts the response to an oral nanoparticulate OVA antigen (second column) in terms of the total serum anti-OVA antibodies at day 21. The bars represent the average+/−SD. OVA-SOL represents the oral application of soluble antigen; OVA/NP-ORL nanoparticulate formulation and NP-ORL are empty nanoparticles (no OVA).

FIG. 6 demonstrates in vivo gene expression. DNA-ID represents intradermal injection of naked DNA solution (plasmid); Lipofect/DNA is DNA complexed with Lipofectamine reagent (Gibco, Gaithersburg, Md.); and NP/DNA is DNA encapsulated in nanoparticles.

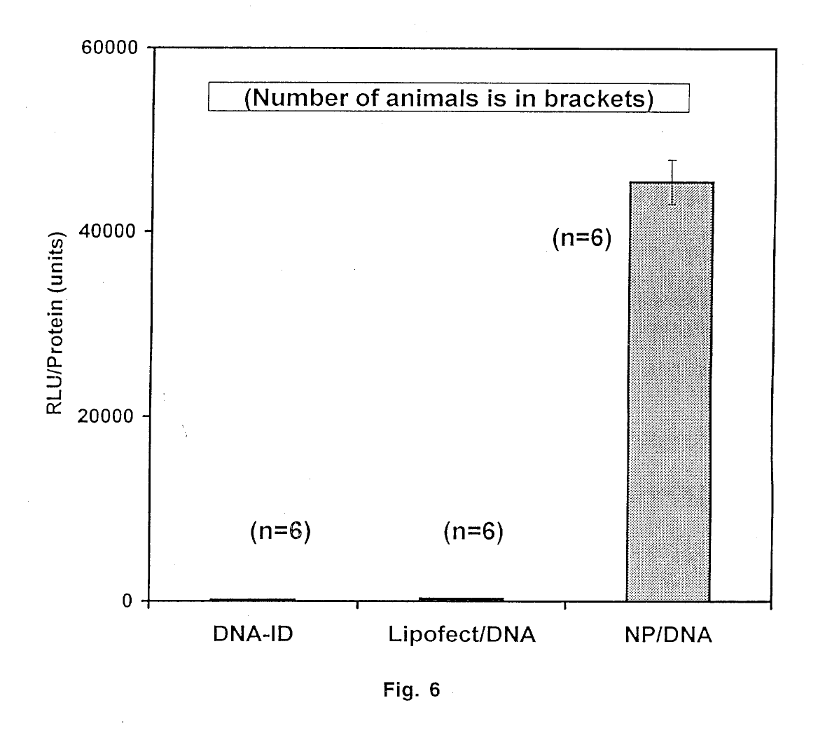

FIG. 7 demonstrates the in vitro release of FGFb from nanoparticles.

### DETAILED DESCRIPTION OF THE INVENTION

In one embodiment of the present invention there is provided a method of making a nanoparticle that is stable in a physiological environment for at least a day where the nanoparticle comprises polyanionic polymers and polycations in a complex useful for drug delivery. The method comprises the step of capturing droplets comprising the polyanionic polymers in a solution that comprises the polycations, or, alternatively, capturing droplets comprising the polycations in a solution that comprises the polyanionic polymers such that the complex structure forming the nanoparticle made by this method increases the stability of the nanoparticle in a physiological environment.

In one aspect of this embodiment the nanoparticles have a polyanionic core and polyanionic/polycationic complex corona with an excess positive charge on the particle periphery. The nanoparticle may comprise a ternary or quaternary complex. In this aspect the polyanionic core may further comprise a protein, an anionic antigen or a nucleic acid incorporated as an integral component of the polyanionic droplets into the polycationic solution to form the nanoparticle. A representative example of nucleic acid is plasmid DNA or an antisense RNA oligonucleotide. Also in this aspect the polyanionic core may further comprise a monovalent or bivalent salt. Examples of such salts are sodium chloride, calcium chloride or sodium sulfate. These salts may be present in a concentration up to about 3 wt %. In a related aspect the nanoparticles may have a polycationic core and a polycationic/polyanionic complex corona with the excess of negative charge on the particle periphery.

In other aspects of this embodiment the polyanionic polymers or the polycationic polymers which may form corona polymers of the nanoparticle may further comprise a charged polymeric surface modifier. The charged polymeric surface modifier has the same charge as the corona polymers. In this aspect the corona polymers and the charged surface modifier are incorporated in one step as an integral component of the nanoparticle. Additionally, the polyanionic polymers or the polycationic polymers may further comprise a nonionic polymeric surface modifier. The nonionic surface modifier is incorporated into the nanoparticle as a steric stabilizer.

In all aspects of this embodiment the polyanionic droplets or the polycationic droplets may be in a mist, may be uniformly sized in a stream or may be in a solution. The polyanions and the polycations may be mixed together in the ratio of from about 1:1 to about 1:4. Additionally, each of the polyanions and each of the polycations are present in concentrations of about 0.01 wt-% to about 2.0 wt-% when making the nanoparticle. Furthermore, the physiological environment may be a physiological media or sera.

In a related aspect to this embodiment there is provided a vaccine comprising the nanoparticle having a polyanionic core and the anionic antigen contained therein. Additionally, this related aspect provides a method of treating an animal to elicit an immune response by administering the vaccine to the animal where the vaccine contains an effective amount of the anionic antigen to elicit the immune response. The vaccine may be administered orally or intravenously. In an example of such immunization orally administered nanoparticles comprising the vaccine are taken up by M-cells in Peyer's patches of the epithelial lining of the upper intestinal tract of the animal resulting in an increase in secretory and systemic antibodies in blood.

In another embodiment of this invention there is provided a method of increasing the entrapment efficiency of a nanoparticle by capturing droplets comprising polyanionic polymers and a salt in a solution comprising polycations, said polyanionic polymers and said polycations sufficient in number to form a ternary or quaternary polymeric nanoparticle structure and forming the nanoparticle structure comprising a polyanionic/salt core and a polycationic/polyanionic complex corona with an excess positive charge on the nanoparticle periphery where the structure of the nanoparticle increases the stability thereof in a physiological environment and thus increasing entrapment efficiency. In all aspect of this embodiment the delivery of the polyanionic droplets, the amounts and concentrations of the polyanions and polycations, the salt and salt concentration comprising the polyanionic core, the physiological environment, and the anionic protein, anionic antigen or nucleic acid further comprising the core are as described supra.

In yet another embodiment of this invention there is provided a nanoparticle comprising a core which itself comprises polyanionic polymers and a salt and a corona surrounding the core which comprises a polycationic/polyanionic complex with an excess positive charge on the nanoparticle periphery such that the polyanionic polymers and said polycations are sufficient in number to form a ternary or quaternary polymeric nanoparticle structure. In this embodiment the salt and the salt concentration comprising the polyanionic core and the anionic protein, anionic antigen or nucleic acid further comprising the core are as described supra. Additionally, the core may comprise a cationic protein in an amount that does not insolubly complex with a polyanionic polymer in the core.

In a related aspect to this embodiment there is provided a method of delivering a protein, an antigen or a nucleic acid to an animal to treat the animal by administering the nanoparticle to the animal such that the nanoparticle contains an effective amount of the anionic antigen or the nucleic acid to effect treatment of the animal. The nucleic acid may be plasmid DNA or an antisense RNA oligonucleotide. In this aspect the nanoparticle and the anionic antigen may comprise a vaccine. The vaccine contains sufficient antigen to elicit an immune response and may be used to immunize and animal. The nanoparticles and vaccines may be adminstered orally or intravenously.

In yet another embodiment there is provided a method of processing reactor content to remove unwanted residual reactants comprising the steps of sedimenting or centrifuging the reactor content; collecting microparticles or the nanoparticles described herein generated in a stirred reactor; rinsing the particles in an excess of water, buffer or cryopreservation solution; separating the suspension by the sedimentation or centrifugation step; repeating the rinsing and separation steps, and reducing the volume of the suspension to about 1/100th of the initial volume.

In a related embodiment there is provided a method for the chemical stabilization of the washed and isolated particles of described supra, comprising the steps of reacting the particles with a crosslinking agent; rinsing the particles in an excess of water, buffer or a cryopreservation solution; separating the particles by sedimentation or centrifugation repeating the rinsing and separation steps, and reducing the volume of the suspension. Representative examples of the crosslinking agent are dextran polyaldehyde, a photocrosslinking polymer and a γ-glutamyl transferase enzyme.

In another related embodiment there is provided a method of cryoprotecting the washed particles described supra by suspending the particles in a cryoprotective solution to form a suspension and lyophilizing the suspension. The cryoprotective solution may be glycerol, trehalose, sucrose, PEG, PPG, PVP, block polymers of polyoxyethylene and polyoxypropylene, block polymers of polyethylene oxide and polyvinylacohol, polymeric 2-methacryloxyethylphosphorylcholine, and water soluble derivatized celluloses. The cryoprotective solution may be in a concentration of 1 wt-% to 10 wt-%.

In still another embodiment of the present invention there is provided a method of adjustment of the biodegradability of polymeric mixtures by contacting an enzyme to a polysaccharide and degrading the polysaccharide at physiological conditions in vivo to degradation products. Examples of the enzyme are alginate-lyase (alginase) and carrageenase for polymer matrices containing alginate or carrageenans.

In still yet another embodiment of the present invention there is provided a method of introducing an adjuvant to potentiate an immunogenic effect by administering the adjuvant as part of a droplet-forming polymeric mixture. The adjuvant may be an aluminum salt enabling to gel certain polysaccharides. Additionally, the adjuvant may be CMC, CS and HV alginate as droplet forming anionic polymers and aluminum sulfate, calcium chloride and a polycationic polymers as a corona forming mixture.

As used herein, the term “reactor” shall refer to an enclosed vessel provided with or without a stirrer, allowing for a reaction to proceed in liquid or gas phases.

As used herein, the term “insoluble submicronic particles” shall refer to particles that remain solid in essentially water-based solutions, such as water, saline, PBS, serum or a physiological buffer.

As used herein, the term “light scattering” or “Tyndall effect” shall refer to light dispersion in many directions, resulting in a slightly milky suspension, visible by a human eye.

As used herein, the term “ultrasonic probe” shall refer to a hollow metallic tube whose tip oscillates many cycles per second as directed by a power imposed upon it.

As used herein, the term “nanoparticle” shall refer to submicroscopic, i.e. less than 1 micrometer in size solid object, essentially of regular or semi-regular shape.

As used herein, the term “corona” shall refer to an insoluble polymeric electrostatic complex composed of internal core polymer(s) and receiving bath polymer(s) molecularly bound in a close proximity.

As used herein, the term “cations” shall refer to a combination of cations, such as, but not limited to, calcium chloride, potassium chloride or aluminum sulfate, and/or polycationic polymers.

As used herein, the term “polycation” shall refer to a polycationic polymer.

As used herein, the term “polyanion” shall refer to a polyanionic polymer.

As used herein, the term “structural (gelling) polymer” shall refer to polymers which can form semi-solid gelled structure by means of a small ion complexing.

As used herein, the term “core polymer” shall refer to a drop-forming polymer which represents an internal or central part of the nanoparticle.

As used herein, the term “charged polymeric surface modifiers” or “electrostatic stabilizers” shall refer to a polyelectrolyte or polymer exhibiting a high charge density and, as such, providing the particle periphery with a high surface charge density, allowing for a strong repulsion force between adjoining particles.

As used herein, the term “nonionic polymeric surface modifier” or “steric stabilizer” shall refer to nonionic, i.e., without charge, polymers with protruding side chains residing on the particle periphery and preventing intimate contact between adjoining particles.

As used herein, the term “cryoprotecting” shall refer to substances used for suspension of particles, which upon their water removal in vacuum allow particles to remain in individual and nonaggregating states.

In the description of the present invention, the following abbreviations may be used: SA-HV, high viscosity sodium alginate; CS, cellulose sulfate; k-carr, kappa carrageenan; LE-PE, low-esterified pectin (polygalacturonic acid); PGA, polyglutamic acid; CMC, carboxymethylcellulose; ChS-6, chondroitin sulfate-6; ChS-4, chondroitin sulfate-4; F-68, Pluronic copolymer; GGT, γ-glutamyl transferase; DPA, dextran polyaldehyde; PVSA, polyvinylsulphonic acid; PVPA, polyvinyl phosphonic acid; PAA, polyacrylic acid; PVA, polyvinylamine; OVA, ovalbumin, C-OVA, cationized ovalbumin; BSA, bovine serum albumin; AG, acacia gum; 3PP, pentasodium tripolyphosphate; PMCG, poly(methylene-co-guanidine) hydrochloride; SH, spermine hydrochloride; PS, protamine sulfate; PEI, polyethyleneimine; PEI-eth, polyethyleneimine-ethoxylated; PEI-EM, polyethyleneimine, epichlorhydrin modified; Q-PA, quartenized polyamide; pDADMAC-co-acrylamide, polydiallyldimethyl ammonium chloride-co-acrylamide; PBS, phosphate-buffered saline; PEG, polyethylene glycol; PPG, polypropylene glycol; PEO, polyethylene oxide; HEC, hydroxyethyl cellulose; ACCP, SA/CS/CaCl₂/PMCG; and ACCSP, SA/CS/SP/CaCl₂/PMCG.

The present invention is a water-based technology. Resulting particles consist of a dense polymeric core matrix, in which a drug, protein, antigen, plasmid DNA, or antisense nucleotide can b e dispersed or dissolved and surrounded by a polymeric shell or corona. The particulate delivery systems have been widely used, but difficulties with biocompatibility, particle strength and the inability to define and modify parameters critical for such delivery vehicles has prevented this technology from achieving its full potential. A typical problem is a use of organic solvents for manufacturing particles, rather loose association of plasmid DNA within a liposome (17) or a low stability of the DNA-spermine complex at physiological conditions (18). In addition, liposomes exhibit a very low incorporation of highly hydrophilic substances, such as DNA or polynucleotide.

Provided herein are new combinations of multicomponent water-soluble polymers which allow for modification of the particle size down to a desirable size, adequate mechanical strength, desirable permeability and surface characteristics, and stability in the presence of salt or sera. The present invention is directed to a composition of matter comprising various polyanion and polycation mixtures and methods of making said. Such compositions can be made using a stirred reactor to mix polyanionic and polycationic compoments therein to form a particle exhibiting increased stability in a physiological environment such as a physiological media or sera.

The increased stability of the particles results in, inter alia, increased entrapment efficiency for a more efficacious delivery of a biomolecule contained within the core of the particle. A particularly usable combination is one of anionically charged antigen or a nucleic acid, such as plasmid DNA or antisense oligonucleotide and SA-HV/CS as the polyanion or a CaCl₂/SP/PMCG complex as the polycation. It is also preferred that in a nanoparticle with a polyanionic core, the core comprises a monovalent or bivalent salt such as sodium chloride, calcium chloride, or sodium sulfate. In a particle having a polycationic core a cationic drug may be incorporated therein.

The particles described herein may be made by providing a stream of uniformly-sized submicron or few micron drops of polyanionic polymer solution by means of a hollow ultrasonic device and collecting these droplets in a stirred reactor provided with a cationic solution. The particles have a polyanionic core and polyanionic/polycationic complex shell (corona) with an excess of the positive charge on the particle periphery. Conversely, a droplets of cationic solution of the requisite size are collected in a polyanionic solution. The particles have a polycationic core and polycationic/polyanionic complex shell (corona) with the excess of negative charge on the particle periphery.

Alternatively, the polyanionic and polycationic solutions are mixed together in the ratio of 1:1 to 1:4 or the same ratio of polycationic to polyanionic solutions may be used and gently stirred for 5-10 minutes. For many combinations of polymers, a spontaneous formation of particles is observed. Still alternatively, streams of uniformly sized submicron droplets of both polyanionic and polycationic solutions are reacted in a gas-phase reactor.

The individual components of the core polyanionic solution of polymers include concentrations of 0.01 wt-% to 0.5 wt-%. In a more preferred composition each component of the polyanions is at a concentration of 0.05 wt-% to 0.2 wt-%. In addition, the individual components of the corona cationic solution are at a concentration of 0.01 wt-% to 0.5 wt-%. In a most preferred. composition, the polycations are at 0.05 wt-% to 0.2 wt-% and calcium chloride at 0.05 wt-% to 0.2 wt-% or potassium chloride at 0.05 wt-% to 0.2 wt-% in case carrageenans are used as anionic polymers.

The individual components of the core cationic solution of polymers and inorganic salts include concentrations of 0.01 wt-% to 2.0 wt-%. In a more preferred composition each component of the polycations and each inorganic salt is at a concentration of 0.05 wt-% to 0.2 wt-%. In addition, the individual components of the corona polyanionic solution are at a concentration of 0.01 wt-% to 0.5 wt-%. In a most preferred composition, the polycations are at 0.05 wt-% to 0.2 wt-%.

Further provided is a method of post-production processing of particles consisting of recovery and washing steps. The reactor content is processed by sedimenting or centrifuging the reactor mixture. The microparticles or nanoparticles are collected as a pellet and rinsed in a large excess of water, buffer, cryopreservation solution, electrostatic or steric stabilizer solution. The resultant suspension is separated by said sedimentation or centrifugation. The rinsing and separation steps are repeatable. Finally the volume of the suspension is reduced to about 1/100th of the initial volume.

Additionally, the particles described herein may b e modified. Methods and compositions for surface modification, crosslinking and/or cryopreservation are provided. The particles may may include a charged polymeric surface modifiers, or electrostatic stabilizers, which is incorporated in one step together with other polymeric components as an integral part of the complex. Similarly, a nonionic polymeric surface modifier, or steric stabilizer, is integrated into the polymer structure via an entrapment. Both classes of surface modifiers are included to prevent particle aggregation upon their further processing. Furthermore, the multicomponent combination of polymers in the particles may be composed of a structural (gelling) polymer and a polymer providing the mechanical strength (crosslinking) and permeability control.

The particles may be stabilized by means of physiological crosslinking agents. The washed and isolated particles are reacted with a crosslinking agent and then rinsed in a large excess of water, buffer or a cryopreservation solution, electrostatic or steric stabilizer solution. The particles are separated via sedimentation or centrifugation; rinsing and separating the particles may be repeated. Finally, the volume of the suspension is reduced. The crosslinking agent is dextran polyaldehyde, a solution of photocrosslinking polymer, or a γ-glutamyl transferase enzyme. The reaction conditions are selected accordingly, but within the physiological realm.

Cryoprotection with concomitant stabilization is provided by means of lyophilization. The washed particles are suspended in a cryoprotective solution and lyophilization of the suspension is performed in a suitable lyophilization apparatus. Such cryoprotective solutions may include glycerol, trehalose, sucrose, PEG, PPG, PVP, block polymers of polyoxyethylene and polyoxypropylene, water soluble derivatized celluloses and some other agents at a concentration of 1 wt-% to 10 wt-%.

In addition, the biodegradation of the particles may be adjusted. A suitable amount of a suitable enzyme is added to a polysaccharide to be degraded. The polysaccharide is broken down at physiological conditions in vivo to degradation products that can be further broken to nonharmful products in animal/human body. The enzyme is provided in quantities allowing for controlled biodegradation in the range of one week to several months. A preferred enzyme is alginate-lyase (alginase) or carrageenase for polymer matrices containing alginate or carrageenans.

The particles described herein are useful for drug delivery. Using a new combination of polymers and new encapsulation technology and generating very small particles, the present invention provides a multicomponent particle formed by polyelectrolyte complexation. In case the drug or targeted biological substance is polyelectrolyte by virtue of its nature, such components become an integral part of the particle core.

It is specifically contemplated that pharmaceutical compositions may be prepared using a drug, protein, antigen, plasmid DNA, or an antisense RNA oligonucleotide, encapsulated in the particles of the present invention. In such a case, the pharmaceutical composition may comprise a drug or other biomolecule or bioproduct and a biologically acceptable matrix. This microparticulate and nanoparticulate technology has been applied as a vehicle for oral delivery of antigen leading to subsequent immunization in vivo. Additionally, those polyelectrolyte complexes with a polyanionic or polyanionic/salt core may be administered intravenously and demonstrate stability in sera.

A person having ordinary skill in this art would be able to determine readily, without undue experimentation, the appropriate concentrations of said biotechnology products, matrix composition and routes of administration of the particles of the present invention. For example, the compositions may be administered orally, intravenously, nasally, rectally or vaginally, through inhalation to the lung, and by injection into muscle or skin or underneath the skin.

Additionally, the particles may comprise an adjuvant to potentiate an immunogenic effect. The adjuvant is preferably aluminum salt enabling to gel certain polysaccharides. Preferably, the particle includes CMC, CS and HV alginate as droplet forming anionic polymers, either individually or in a mixture, and aluminum sulfate or any other water soluble aluminum salt, calcium chloride and suitable polycationic polymers as a corona forming mixture. Furthermore, mucoadhesive polymers may be added to the corona-forming bath to provide for sticking properties in relation to mucosal areas in the animal/human body. Examples of mucoadhesive polymers are heparin, ChS-4, ChS-6, carrageenans, xanthan, gellan, pectin, gelatin, CS, CMC, and chitosan.

The following examples are given for the purpose of illustrating various embodiments of the invention and are not meant to limit the present invention in any fashion.

### EXAMPLE 1

Particle Production Process

Particles may be made in a stirred reactor. In a more preferred mode, such a reactor is filled with a cationic solution. A mist of anionic droplets was generated by means of a hollow ultrasonic probe and introduced into the cationic solution residing in the reactor or receiving bath. Typically, 1-2 ml of anionic solution is extruded into 20 ml of cationic solution in a batch mode, resulting in a nonstoichiometric complex with an excess of cationic charge on the particle periphery. Instantly, insoluble submicronic particles are formed as evidenced by a light scattering or Tyndall effect. The reaction time can be adjusted. Typically, 1-2 hours is sufficient for particle maturation. This is due to their thermodynamic instability, large surface area and surface free energy. The size can be measured by means of a Malvern ZetaMaster (Malvern, UK).

The composition and combinations of anionic polymer mixture as well of the cationic receiving bath is essential to allow for adjustments in particle size, shape and uniformity. Conversely, droplets can be made from polycationic solution and the receiving bath contains then a polyanionic solution. Alternatively, the polyanionic and polycationic solutions are mixed together in the ratio of 1:1 to 1:4 which is the same ratio of polycationic to polyanionic solutions in the converse mode and gently stirred for 5-10 minutes without the employment of the ultrasonic probe. For many combinations of polymers, a spontaneous formation of particles is observed.

Still alternatively, a continuous flow reactor system was constructed, composed of a continuous stream of submicron size drops of the core polyanionic solution, by means of a hollow ultrasonic device and a continuous stirred reactor filled with a corona cationic solution and provided with an inflow and outflow of this solution. The core solution is continuously introduced into the corona solution; the ratio of droplet for the core to corona-forming solution flow rates is adjusted to result in a nonstoichiometric polymeric complex. It is typically 1:20 to 3:20.

Still alternatively, two polymeric solutions, for the core and for the corona are allowed to react in a gas tubular reactor in the form of a mist generated by two separate hollow ultrasonic devices. The product is easily separated because of large differences between the particle and air densities.

### EXAMPLE 2

Particle development: Polymer screening

Many of thirty-five combinations of polymers resulting from a multicomponent membrane encapsulation system can be suitable for generation of small microparticles and nanoparticles. To develop new polymeric combinations of water-soluble polyanions and polycations, the instrumentation and process described in Example 1 was used. The criteria for selection was formation of submicroscopic particles as demonstrated by the Tyndall effect. Individual polymers were tested for biocompatibility using an in vitro culture system with rat insulinoma cells (RIN 1046-38 cells, American Type Culture Collection, Rockville, Md.). Preferred combinations are listed in TABLE I. In contrast to cell microencapsulation systems requiring a combination of polymers resulting in a gelled interaction for sufficient membrane strength, small microparticulate and nanoparticulate systems result, in addition to the above, also from interactions resulting in a precipitated complex. Out of three different reaction products resulting from interactions of water-soluble polymers-polyelectrolytes of an opposite charge, soluble complex is the least desirable, not leading to particles. Precipitated complex is acceptable as long as it remains insoluble after its formation. The electrostatic insoluble complex is the best option. Both precipitated and electrostatic complexes are desirable for micro- and nanoparticle formation.

TABLE I
Multicomponent particulate systems

| Anionic components | Cationic components |
|---|---|
| SA-HV/3PP | Chit/calcium chloride |
| SA-HV/LE-pectin | BSA/calcium chloride |
| LB-pectin | PEI/calcium chloride |
| ChS-4/SA-HV | Gelatin A/calcium chloride |
| LE-pectin/SA-HV | Gelatin A/calcium chloride |
| Acacia/SA-HV | Gelatin A/calcium chloride |
| κ-carr/SA-HV | BSA/calcium chloride/potassium chloride |
| CS/SA-HV | Chit/calcium chloride |
| Sodium sulfate/SA-HV | Chit/calcium chloride |
| Gelatin B/SA-HV | Chit/calcium chloride |
| ChS-6/SA-HV | Chit/calcium chloride |
| ChS-4/SA-HV | Chit/calcium chloride |
| Gellan/SA-HV | PLL/calcium chloride |
| LE-pectin/SA-HV | Q-PA/calcium chloride |
| SA-HV/CS | Chit/calcium chloride |
| SA-HV/PGA | Chit/calcium chloride |
| CS/PGA | Chit/calcium chloride |
| Xanthan/gellan | PLL/calcium chloride |
| Xanthan/CS | PEI-eth/calcium chloride |
| Xanthan/κ-carr | PEI-eth/calcium chloride/potassium chloride |
| Xanthan/gellan | PEI-eth/calcium chloride |
| Xanthan/CS | PEI-EM/calcium chloride |
| Xanthan/CMC/ | pDADMAC-co-acrylamide/ |
| | aluminum sulfate |
| CS/SA-HV | PVA/calcium chloride |
| CS/CMC | PVA/calcium chloride/aluminum sulfate |
| CS/gellan | PVA/calcium chloride |
| CMC/gellan | PVA/aluminum sulfate/calcium chloride |
| CS/CMC | Q-PA/calcium chloride |
| CS/xanthan | Q-PA/calcium chloride |
| CS/κ-carr | Q-PA/calcium chloride |
| CS/gellan | Q-PA/calcium chloride |
| CMC/xanthan | Q-PA/calcium chloride |
| CMC/κ-carr | Q-PA/calcium chloride |
| CMC/gellan | Q-PA/calcium chloride |
| Xanthan/κ-carr | Q-PA/calcium chloride |
| Xanthan/gellan | Q-PA/calcium chloride |
| CS/CMC | Polybrene |
| CS/xanthan | Polybrene |
| CS/κ-carr | Polybrene |
| CS/gellan | Polybrene |
| CMC/xanthan | Polybrene |
| CMC/κ-carr | Polybrene |
| CMC/gellan | Polybrene |
| Xanthan/κ-carr | Polybrene |
| Xanthan/gellan | Polybrene |
| PVPA/SA-HV | Chit/calcium chloride |
| PVSA/SA-HV | Chit/calcium chloride |
| PVPA/CS | Chit/calcium chloride |
| PVSA/CS | Chit/calcium chloride |
| SA-HV/3PP | PVA/calcium chloride |
| CS/3PP | PVA/calcium chloride |
| CMC/3PP | PVA/calcium chloride |
| CMC/3PP | PVA/calcium chloride/aluminum sulfate |
| Gellan/3PP | PVA/calcium chloride |
| Xanthan/SA-HV | PLL/SP |
| SA-HV/gellan | SH/PMCG |
| SA-HV/CS | SH/PMCG |
| SA-HV/gellan | PH/PMCG |
| SA-HV/CS | PH/PMCG |
| SA-HV/gellan | Polybrene/PMCG |
| SA-HV/CS | Polybrene/PMCG |
| κ-carr | PS/calcium chloride/potassium chloride |
| κ-carr/SA-HV | PS/calcium chloride/potassium chloride |
| κ-carr | SP/calcium chloride/potassium chloride |
| κ-carr/SA-HV | SP/calcium chloride/potassium chloride |
| κ-carr | Polybrene/calcium chloride/potassium chloride |
| κ-carr/SA-HV | Polybrene/calcium chloride/potassium chloride |
| κ-carr/heparin | PS/potassium chloride |
| κ-carr/heparin | Polybrene/potassium chloride |
| κ-carr/heparin | SH/potassium chloride |
| CS/heparin | PS/calcium chloride/potassium chloride |
| CS/heparin | Polybrene/calcium chloride/potassium chloride |
| CS/heparin | SH/calcium chloride/potassium chloride |
| PVSA/SA-HV | Chit/calcium chloride |
| κ-carr/gellan | PVA/calcium chloride |
| SA-HV/gellan | PVA/calcium chloride |
| PAA/SA-HV | Chit/calcium chloride |
| PAA/CS | Chit/calcium chloride |
| PAA/gellan | Chit/calcium chloride |
| PAA/κ-carr | Chit/calcium chloride |
| SA/CS | calcium chloride/PMCG |
| SA/CS | calcium chloride/SH/PMCG |

### EXAMPLE 3

Nanoparticle 1

This particle was generated using a droplet-forming polyanionic solution composed of 0.1 wt-% HV sodium alginate (SA-HV) and 0.05 wt-% chondroitin sulfate C (ChS-C) in water and corona-forming polycationic solution composed of 0.1 wt-% spermine hydrochloride (SH), 0.01 wt-% poly-L-lysine hydrochloride (PLL) and 0.2 wt-% calcium chloride in water. The chemicals used were: high viscosity sodium alginate (SA-HV) from Kelco/Merck (San Diego, Calif.) of average molecular weight 4.6×10⁵; chondroitin sulfate-6 (ChS-6) from Sigma (St. Louis, Mo.); spermine hydrochloride (SH); poly-L-lysine (PLL), of average molecular weight 4.5×10⁴; and calcium chloride. The ratio of droplet- to corona-forming reactants was 1.0:20. The particles were instantaneously formed in a batch system, allowed to react for 2 hours and their size and charge evaluated in the reaction mixture. The average size was 280 nm and the average charge 20.1 mV. Particles remained stable as individual entities during four week period at 4° C. The size of particles tended to increase upon their processing (washing in saline or water), if not stabilized.

### EXAMPLE 4

Nanoparticle 2

This particle was generated using a droplet-forming polyanionic solution composed of 0.1 wt-% HV sodium alginate (SA-HV) and 0.1 wt-% CS in water and corona-forming polycationic solution composed of 0.1 wt-% PMCG hydrochloride, and 0.2 wt-% calcium chloride in water. The polymers used were: cellulose sulfate, sodium salt (CS) from Janssen Chimica (Geel, Belgium) having an average molecular weight 1.2×10⁶; and poly(methylene-co-guanidine) hydrochloride (PMCG) from Scientific Polymer Products, Inc. (Ontario, N.Y.), with average molecular weight 5×10³. The ratio of droplet- to corona-forming reactants was 1.5:20. The particles were instantaneously formed in a batch system, allowed to react for 1 hour and their size and charge evaluated in the reaction mixture. The size distribution was bimodal, with two categories: average size 3-5 μm and particles with average size <1 μm (Tyndall effect). The average charge 15.2 mV. Particles remained stable for 3 week observation period at 4° C. When these particles were further washed with saline or water, their size increased dramatically, unless stabilized.

### EXAMPLE 5

Nanoparticles with Integrated Anionic Immunogen

These particles were generated using a droplet-forming polyanionic solution composed of 0.1 wt-% AG, 0.1 wt-% OVA and 0.1 wt-% SA-HV in water, adjusted to pH 4.0 and corona-forming polycationic solution composed of 0.2 wt-% BSA, and 0.2 wt-% calcium chloride in water, also adjusted to pH 4.0. The OVA was used as a model anionic antigen. The ratio of droplet- to corona-forming reactants was 1:20. The polymers used were: acacia gum (AG) from Sigma, average molecular weight 4.5×10⁵, isoelectric point (pI) 4.0; egg ovalbumin (OVA) from Sigma, average molecular weight 4.2×10⁴, pI 4.6; and bovine serum albumin (BSA) from Sigma, with average molecular weight 6.7×10⁴, pI 5.4. The particles were instantaneously formed, allowed to react for 1 hour and their size and charge evaluated in the reaction mixture. The average size was 430 nm and the average charge 15.5 mV. Particles remained stable for 4 week observation time at 4° C. When these particles were further washed with saline or water, they slowly dissolved over a period of few days, unless stabilized by crosslinking.

### EXAMPLE 6

Nanoparticles with Integrated Cationic Immunogen

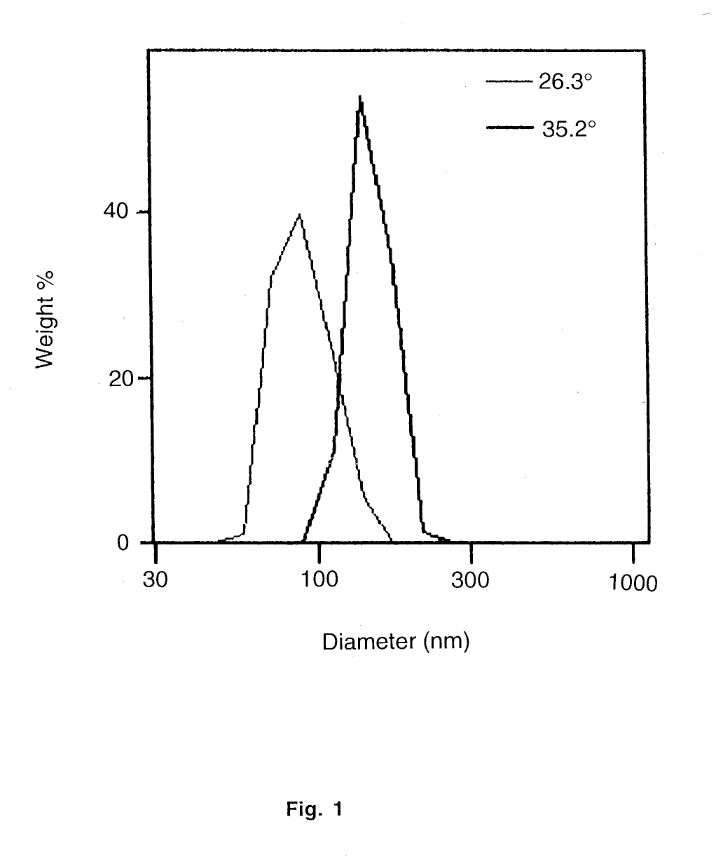

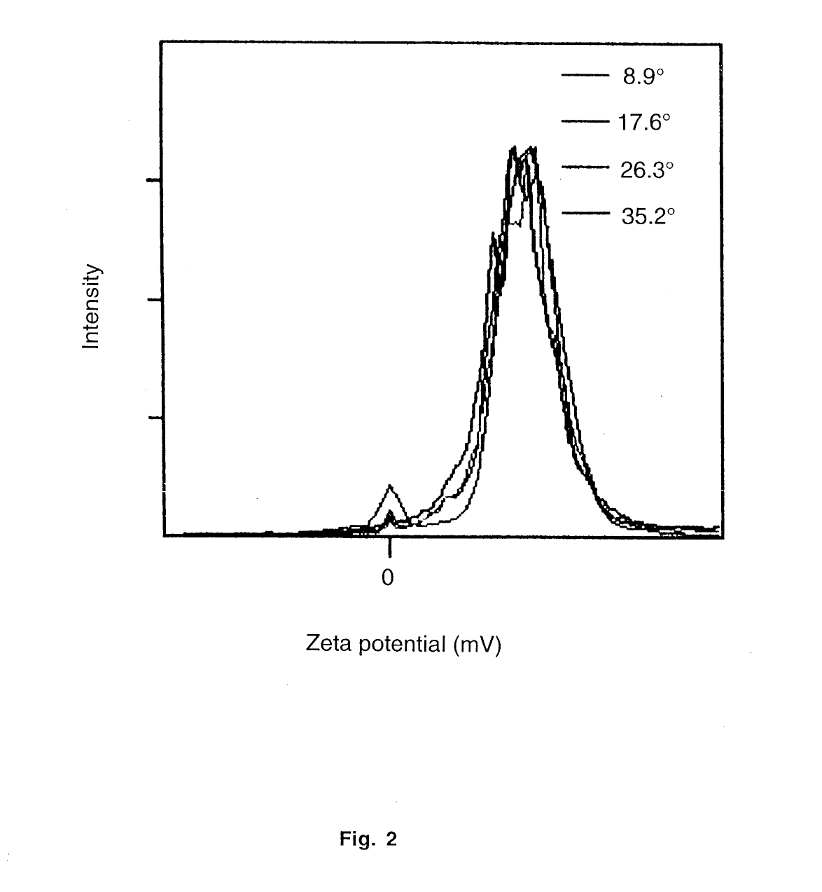

These particles were generated using a droplet-forming polycationic solution composed of 0.1 wt-% Chit, 0.1 wt-% C-OVA and 0.2 wt-% calcium chloride in water, and corona-forming polyanionic solution composed of 0.2 wt-% PGA, and 0.1 wt-% SA-HV in water. The C-OVA was used as a model cationic antigen. The ratio of droplet- to corona-forming reactants was 1:20. The polymers used were: chitosan glutamate Protasan HV (Chit) from Pronova Biopolymers (Drammen, Norway), average molecular weight 7.5×10⁵; cationized ovalbumin (C-OVA), synthesized in-house according to published procedure (19) average molecular weight 4.2×10⁴, pI 10.4; and pentasodium tripolyphosphate (3PP) from Sigma. The particles were instantaneously formed, allowed to react for 1 hour and their size and charge evaluated in the reaction mixture. The average size was 220 nm (FIG. 1) and the average charge −17.0 mV (FIG. 2). Particles remained stable for 5 week observation time at 4° C.

### EXAMPLE 7

Electrostatic Surface Stabilization of Nanoparticles

These particles were generated using a droplet-forming polyanionic solution composed of 0.1 wt-% SA-HV in water, and corona-forming polycationic solution composed of 0.05 wt-% PLL in water. The ratio of droplet- to corona-forming reactants was 1:20. The particles were instantaneously formed, allowed to react for 1 hour and their size and charge evaluated in the reaction mixture. The average size was 220 nm and the average charge 25.4 mV. When particles were washed in water or in saline, they aggregated and formed patches floating on the top of solution. When 0.05 wt-% BSA solution (pH 5.0) was used for successive washing and resuspending, the original size remained unchanged.

### EXAMPLE 8

Steric Surface Stabilization of Nanoparticles

These particles were generated using a droplet-forming polyanionic solution identical to that used in Example 4, with a n additional 0.1 wt-% F-68 added. The corona-forming polycationic solution was same as in Example 4. Pluronic F-68 (BASF, Mount Olive, N.J.) is a water soluble nonionic block polymer composed of polyoxyethylene and polyoxypropylene segments. In contrast to particles generated in Example 4, particle size remained unchanged during the washing steps. Similar results were obtained for other nonionic block co-polymers (20-21) Similar results also were obtained with a copolymer of polyethylene glycol and polyvinylalcohol (BASF Corp, USA) and with a polymeric 2-methacryloxyethylphosphorylcholine (NOF Corp, Japan).

### EXAMPLE 9

Direct Use of the Reaction Product for Oral Antigen Delivery

These particles were generated using droplet-forming polymeric combination identical to that used in Example 4, except the polyanionic mixture had additional polymer, 0.1 wt-% OVA. The reaction mixture, after 2 hour maturation time, was applied as such, without washing, into experimental animals (male Sprague-Dawley adult rats (200-250 g wt., 12-15 weeks old, Harlow). About 1 mg of dry weight of nanoparticles in the reaction mixture has been administered orally into the stomach of each animal. The nonloaded (no OVA) nanoparticles were also administered to control animals, in addition to a soluble antigen (OVA). ELISA assay of secretory IgA and serum IgG antibodies was carried out as described (22). Primary and secondary immunization protocol was used, consisting of three successive days at week 0 and week 4. Immunizations with OVA-nanoparticles resulted in dramatically greater levels of both secretory and serum antibodies (about 30-50 times) than those found with the soluble antigen. In another set of experiments nonloaded nanoparticles (no OVA) were separately tested in simulated gastric (pH 2) and intestinal (pH 8.3) solutions for their stability. The nanoparticles remained stable during the observation period of one week. However, a separate experiment revealed that OVA-loaded nanoparticles released about 40% OVA in 2 hours at pH 2 and continued to release thereafter.

### EXAMPLE 10

Particle Recovery

Particles were separated as in Examples 4 and 5. Average size was 390 nm and average charge 15.9 mV. After a 2 hour maturation time, particles were centrifuged at 15° C. for 15 minutes at 10,000 g in a refrigerated Beckman centrifuge L5-50 (Beckman Instruments, Fullerton, Calif.). Next, the supernatant was carefully aspirated off by means of pipette without disturbing the layer of particles at the bottom of 35 ml centrifuge tubes. The sediment was then resuspended in 1 ml water by repeated pipetting in and out, tubes filled up to 35 ml with water and centrifuged again. After removing the supernatant, a dense suspension of particles (1 ml) was evaluated for size and charge. The average size was 450 nm and average charge +10.2 mV (at pH 6.8).

### EXAMPLE 11

Continuous Production of Nanoparticles

These particles were generated using a droplet-forming polyanionic solution containing 0.1 wt-% 3PP, 0.1 wt-% kappa-carrageenan and 0.3 wt-% OVA in water, and corona-forming solution composed of 0.05 wt-% chit, 0.1 wt-% CaCl₂ and 1 wt-% F-68 in water. The droplet-forming solution was continuously fed at flow rate 1, 1.5 and 2 ml/min into a continuous stirred tank reactor of 50 ml working volume maintained at flow rate of 20 ml/min by core-forming solution. The reactor was operated for 10-20 min to reach a steady-state. Samples of reaction mixture were directly analyzed for size and charge and are shown in Table II. Data on size and charge obtained between 10 and 20 min of continuous operation indicated that the product quality can be easily maintained, as judged by invariance in size and charge. Separately, large volumes of reaction mixture collected at. each flow rate of droplet-forming solution were processed by centrifugation and washing. The size and charge between the direct and processed samples were not significant. The continuous production allows for independent adjustment in size and charge by varying the OVA and F-68 concentrations (data not shown).

TABLE II
Size and charge of continuously produced nanoparticles

| | Core | polymer flow | rate (ml/min) |
|---|---|---|---|
| | 1.0 | 1.5 | 2.0 |
| Size (nm) | 350 | 457 | 655 |
| Charge (mV) | 45.1 | 38.2 | 30.3 |

### EXAMPLE 12

Crosslinking of Nanoparticles

These particles were generated using a droplet-forming polyanionic solution composed of 0.0125 wt-% HMP (Sigma), 0.025 wt-% gellan (Kelco), 0.025 wt-% SA-HV and 12 mg tetanus toxoid antigen (TT) (Connaught Labs., Swiftwater, Pa.) in water, and corona-forming polycationic solution composed of 0.075 wt-% PMCG, 0.05 wt-% CaCl₂ and 2 wt-% F-68 in water. The ratio of droplet- to corona-forming reactants was 3.5:20. Particles were prepared and processed as in Example 10. A portion of water-washed particles was resuspended in 0.01 wt-% dextran polyaldehyde solution (DPA, CarboMer, Westborough, Mass.), average molecular weight 4×10⁴ and incubated in a bicarbonate buffer (pH 8.3) at 37° C. for 15 minutes. Another portion of washed particles was treated with 0.01 wt-% γ-glutamyl transferase (GGT, Sigma) in TRIZMA (Sigma) buffer (pH 8.5) with 10 mM calcium chloride at 20° C. for 30 minutes. Another portion was treated with 0.1 wt-% solution of polyvinyl alcohol bearing styrylpyridinium group which was synthesized in-house (23) and exposed to a visible light source, i.e., a halogen lamp with a cut-off UV filter. Following these crosslinking steps, particles were rinsed in a large excess of water, let sediment and resuspended in a small volume of water. Particles stabilized via such crosslinking remained stable for observed 3 weeks at 4° C., compared to 3-5 days without such treatment.

In another set of experiments, particles were prepared as above but the droplet-forming solution contained an additional polymer, PDA. The concentrations used were 0.00014 wt-%, 0.0007 wt-% and 0.0014 wt-%. Washed particles were resuspended in a bicarbonate buffer (pH 8.3), incubated for 15 min at 37° C. and subsequently washed again. The size and charge of crosslinked particles was not substantially different from those without the crosslinking. A portion of particles was incubated in a Tris buffer (pH 1.85) for 1.5 hour and released protein (TT) was assayed in the supernatant by means of the Pierce method (Table III). The average exposure time 1.5 hours represents, approximately, the residence time of particles in the stomachs of experimental animals.

TABLE III
TT release as affected by crosslinking

| Concentration of PDA | Protein release (% total) |
|---|---|
| 0 (no crosslinking) | 29.5 |
| 0.00014 wt-% | 11.8 |
| 0.0007 wt-% | 10.4 |
| 0.0014 wt-% | 7.6 |

### EXAMPLE 13

Cryoprotection of Nanoparticles

Particles were prepared as in Example 5. First, they were separated by centrifugation at 10,000 x g and then rinsed in water and resuspended in a solution containing a cryoprotective agent. A concentrated suspension of particles was then frozen in a mixture of ethanol-dry ice and lyophilized thereafter using a lyophilization apparatus (The Virtis Co., Gardiner, N.Y.), under a vacuum. In this case, only two cryoprotective agents were tested on two portions of particulate suspension: 2 wt-% PEG (Sigma, average molecular weight 8×10³, and 2 wt-% HEC (Scientific Polymers Products). The resulting product was particulate and easily resuspendable in water. The average size of particles increased from the original 425 nm to 625 nm, or 450 nm to 590 nm, respectively. Similar results were obtained with trehalose as a cryoprotective agent.

### EXAMPLE 14

Adjustment of Nanoparticle Biodegradation

Particles were prepared as in Example 1, except that the core polymeric mixture was adjusted to allow for a slow degradation when applied in vivo. A purified alginase-lyase (alginase) was obtained courtesy of N. L. Schiller (24) and used at levels of 1 and 10 μg/L in the core solution. Particles were prepared, washed in water and incubated 20 minutes at 37° C. in a solution consisting of: TRIS buffer (pH 8.5), 9 mM magnesium chloride, 0.5 M sodium chloride. As a criterion of biodegradation, a time to achieve a visible breakdown of particles was noted. Particles containing 1 μg/L disintegrated in 32 days, particles containing 10 μg/L in 4 days. The control particles (no enzyme) remained intact even after 6 weeks.

### EXAMPLE 15

Use of Nanoparticles for Immunization of Animals

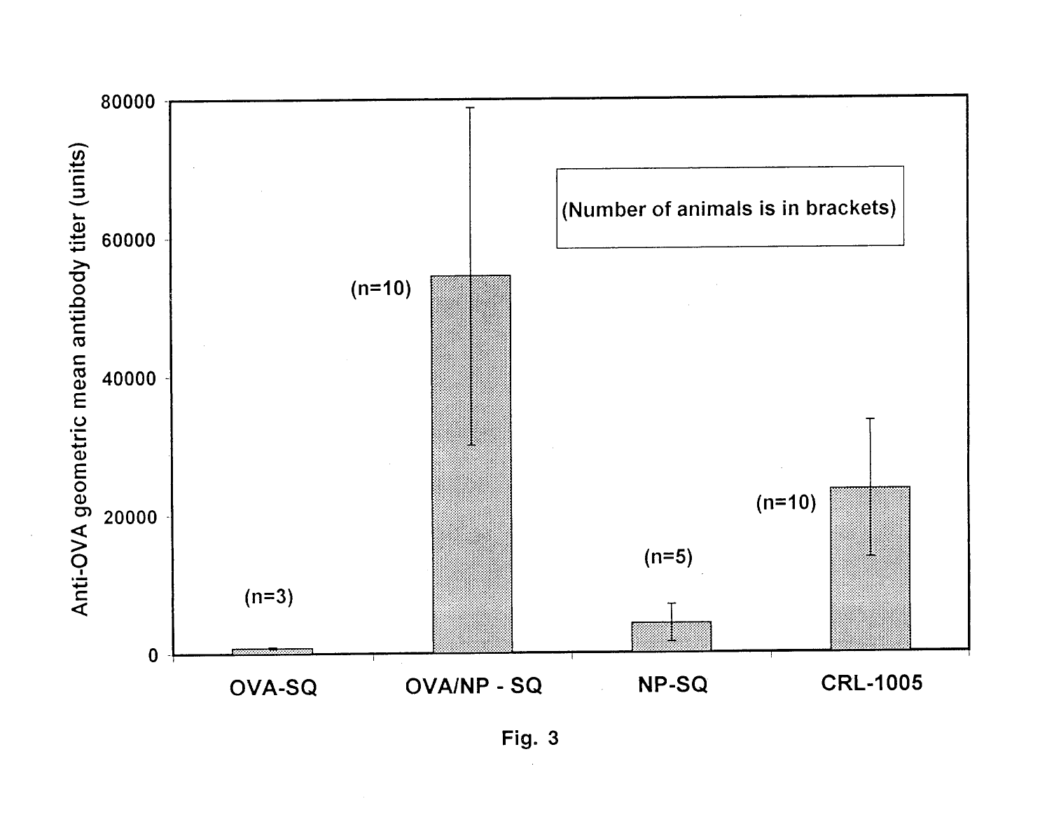

Particles were prepared as in Example 11, stabilized by cross-linking with PDA, as specified in Example 12 (PDA concentration used was 0.001 wt-%), and applied subcutaneously in animals, as specified in Example 9. Results were similar to those reported in Example 9. Total serum anti-OVA antibody titers are presented in FIG. 3. In this figure, OVA-SQ represents the application of soluble antigen OVA, OVA/NP-SQ a subcutaneous application of nanoparticles loaded with OVA, NP-SQ a subcutaneous application of empty (no OVA) nanoparticles (negative control) and CRL-1005 (Vaxcel, Norcross, Ga.) was a positive control (OVA formulated with help of a polymeric adjuvant similar to those mentioned in Example 8). The values are presented in FIG. 3 as the average bar height (n=number of animals)+/−SD (standard deviation).

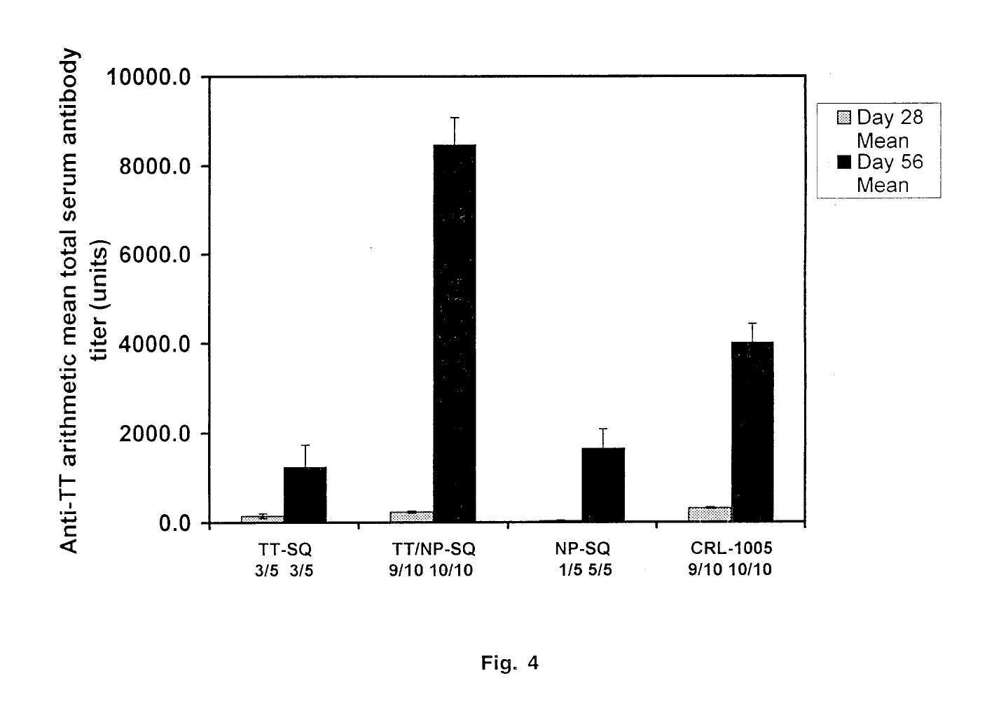

For OVA-SQ, only 3 out of 10 animals per group responded (the others failed to respond at all). For OVA/NP-SQ, all 10 responded. For NP-SQ, 5 out of 10 responded, and for CRL-1005, all 10 responded. Clearly, the OVA entrapped in the nanoparticles is the most effective at eliciting a response. Similar results were obtained with the TT antigen, represented in tabular form in Table IV and graphically in FIG. 4.

TABLE IV
Response to subcutaneous nanoparticulate TT antigen

| | | TT-SQ | TT/NP-SQ | NP-SQ | CRL-1005 |
|---|---|---|---|---|---|
| Day | Mean | 146.0 | 230.0 | 34.0 | 319.0 |
| 28 | | | | | |
| | SD | 53.0 | 22.0 | 13.0 | 22.0 |
| Day | Mean | 1243.0 | 8467.0 | 1646.0 | 3998.0 |
| 56 | | | | | |
| | SD | 487.0 | 614.0 | 431.0 | 427.0 |

### EXAMPLE 16

Use of Aluminum Adjuvant

Nanoparticles were generated using droplet-forming polyanionic mixture composed of 0.1 wt-% CMC and 0.05 wt-% .CS in water and corona-forming polycationic mixture composed of 0.2 wt-% aluminum sulfate and 0.1 wt-% PMCGin water. Chemicals used were: aluminum sulfate (Sigma) and carboxymethylcellulose (brand 7MF, CMC, Aqualon/Hercules, Wilmington, Del.), having medium molecular weight. Aluminum sulfate was incorporated into the nanoparticles as it is known to potentiate antigenic response (25). Particles formed instantaneously and their. average size was 225 nm and average charge 25.4 mV. They were very stable at washing and further processing with no aggregation.

### EXAMPLE 17

Generation of Nanoparticles for Gene Transfer

These particles were generated using a droplet-forming polyanionic solution composed of 0.05 wt-% SA-HV, 0.05 wt-% CS and 0.008 wt-% pCEPluc plasmid in water, and corona-forming olycationic solution composed of 0.05 wt-% SH, 0.065 wt-% PMCG, 0.05 wt-% CaCl₂ and 1.0 wt-% F-68 in water. The latter solution was used as a plasmid condensing agent. pCEPluc is plasmid with a CEP promoter, covalently linked to a luciferase gene as a reporter gene. This plasmid was expressed in a bacterium, grown in a culture and isolated in-house. The ratio of droplet- to corona-forming reactants was 1:10. For particle generation, a special glass double-nozzle atomizer was used. The droplet-forming solution was applied in the internal nozzle, while the air was used to strip particles off the internal nozzle and atomize them into submicron-range size using an internal nozzle. The droplets were then collected in the corona-forming solution. Such device was used because the DNA molecule is sensitive to sonication and can be substantially damaged. The particles were separated by centrifugation and washed. Their size and charge were 190 nm and +24.0 mV, respectively. These particles exhibited an expression of luciferase enzyme in several in vitro cell culture lines.

### EXAMPLE 18

Nanoparticles Loaded with a Cationic Drug

While the preparation of particles with uncharged or anionically charged drugs can be prepared in a similar way as described in Example 5, with replacement of OVA by such a drug, nanoparticles with integrated cationic drug are prepared by a reverse encapsulation, similar to Example 6. These particles are generated using a droplet-forming polycationic solution composed of 0.05. wt-% chit, 0.05 wt-% PVA (15k, Air Products and Chemicals, Allentown, Pa.), 0.05 wt-% CaCl₂ and 0.1 wt-% gentamycin sulfate (Sigma) in water (adjusted to pH 5.0) and corona-forming polyanionic solution composed of 0.1 wt-% 3PP and 1 wt-% F-68 in water. The ratio of droplet- to corona-forming reactants was 1.5:20 or 2:20. The particles were instantaneously formed, allowed to react one hour and their size and charge evaluated after a standard separation and washing. The average size was 86 nm and the average charge was +34.4 mV.

### EXAMPLE 19

Slow Release of Substances

Large microcapsules were prepared by means of an atomization technique. Capsules were of an average size of 350 μm and capsule chemistry was similar to that of Example 4. To measure a release rate, capsules were equilibrated with a tracer solution overnight. A capsule pellet (0.5 ml) was then placed in 5 ml test buffer (PBS) on a shaker and successive aliquots were taken and analyzed. The tracer quantity was assayed using the methods described below. Insulin (Sigma) and OVA were used as tracers. Insulin was assayed by a RIA method by means of Coat-A-Count Insulin Detection Kit (diagnostic Products Corp., Los Angeles, Calif.) and OVA by a protein assay (Bradford) method (Bio-Rad, Hercules, Calif.). The permeability was assessed via an efflux method [11]. Results are listed in Table V. As shown in Table V, permeability can be controlled by means of cation concentration (PMCG, calcium chloride) and by reaction time.

TABLE V
Permeability data for insulin and OVA

| | | | | Zeroeth order | |
|---|---|---|---|---|---|
| System | | Reaction time | | constant | (1/min) |
| Anion blend | Cation blend | (min) | rate | insulin | OVA |
| SA-HV 0.6% | PMCG 1% | 1.0 | | 0.29 | 0.18 |
| CS 0.6% | CaCl₂ 1% | | | | |
| SA-HV 0.6% | PMCG 2% | 0.5 | | 0.07 | 0.01 |
| CS 0.6% | CaCl₂ 1% | | | | |
| SA-HV 0.6% | PMCG 1% | 1.5 | | 0.32 | 0.25 |
| CS 0.6% | | | | | |

### EXAMPLE 20

Use of Mucoadhesive Polymers

For antigen delivery to mucosal areas, it is desirable that the outer particulate surface has mucoadhesive properties. Many polymers listed for multicomponent systems (TABLE I) are believed to be mucoadhesive (heparin, ChS-4, ChS-6, carrageenans, xanthan, gellan, pectin, gelatin, CS, CMC, chitosan). In addition, other polymers can be considered (crosslinked PAA, polymethacrylic acid, hyaluronic acid and collagen). Many uncharged polymers can be incorporated (as an additional component) into the multicationic component (corona) system: HPC, HEC, scleroglucan (SG), polyhydroxymethacrylate (pHEMA), PVP, PVA, PEO, PEG and copolymers of the above. The listed polymers can be used as mucoadhesive polymers as well as polymers exhibiting steric surface stabilization effect (Example 8). Some special substances can also be added to the list: mussel adhesive protein, plant and bacterial lectins and other specialty mucoadhesive polymers. All mucoadhesive polymers can be used in the corona forming mixture in the range of 0.01 to 0.2 wt % in the receiving bath. Thus, the mucoadhesive polymers become integral part of the micro- and nanoparticulate system at processing.

### EXAMPLE 21

Nanoparticles for Oral Delivery of Antigen

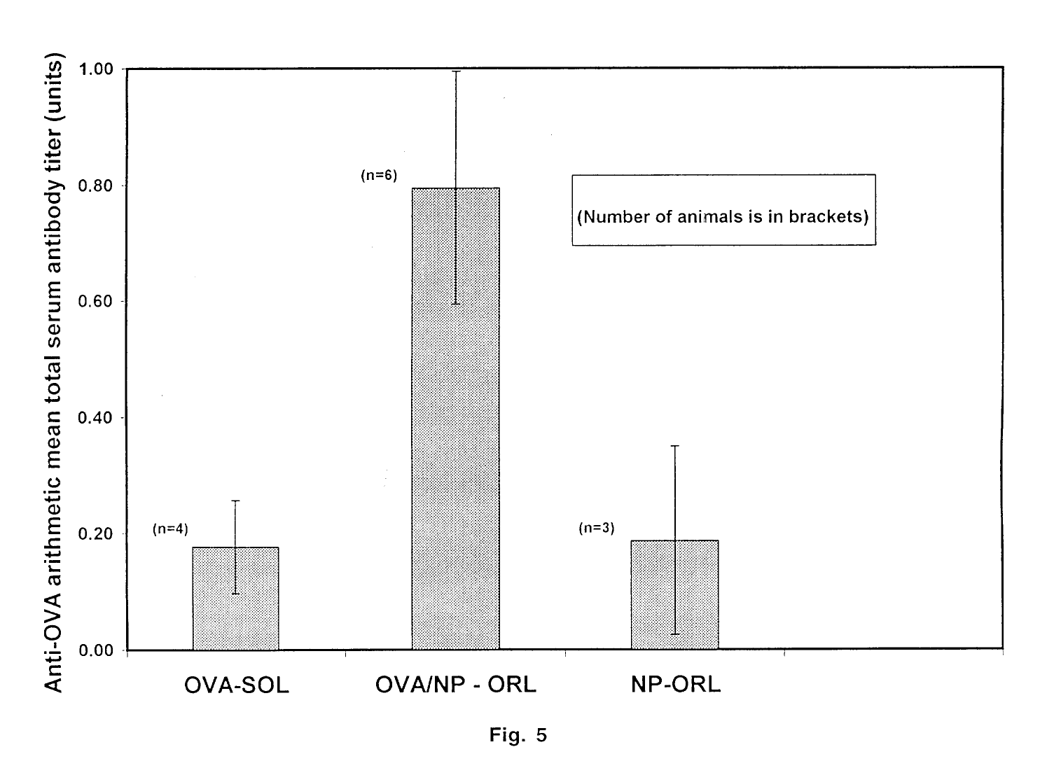

These particles were generated using a droplet-forming 20 polyanionic solution composed of 0.05 wt-% SA-HV, 0.05 wt-% CS, 0.4 wt-% OVA and 0.012 wt-% PDA in water, and Corona-forming polycationic solution composed of 0.05 wt-% SH, 0.065 wt-% PMCG, 0.05 wt-% CaCl₂ and 1.0 wt-% F-68 in water. Particles were separated at 15,000 g and incubated in a bicarbonate buffer to carry the crosslinking reaction as described in Example 12. They were again washed and centrifuged at 15,000 g. The average size and charge were evaluated to be 210 nm and 35.1 mV, respectively. The nanoparticles were then introduced orally into the experimental mice (C57/BL, Harlan, Indianapolis, Ind.). The immunizations were carried at day 0, 7 and 14. At day 21, blood was collected and assayed for the total serum anti-OVA antibodies. Results are summarized in FIG. 5. The immunizations with OVA-nanoparticles resulted in greater levels of serum antibodies than those found with soluble antigen.

### EXAMPLE 22

Use of Nanoparticles for Gene Delivery in vivo

These particles were generated using a droplet-forming anionic solution containing 0.025 wt-% pCEPluc plasmid in water, and corona-forming cationic solution composed of 0.05 wt-% Tetronic 904 (BASF) in water. The ratio of droplet- to corona-forming reactants was 1:1. Two reactants were simply mixed together (polyanion added to the polycation) to form nanoparticles. Their size and charge were 190 nm and +24.0 mV, respectively. The particles were resuspended in isotonic 5 wt-% glucose solution and injected intradermally into 5 experimental animals (see Example 9), 0.1 ml per site. Six sites have been applied per animal. Each animal had 2 negative controls (5 wt-% glucose) and two positive controls (5 wt-% glucose, 0.025 wt-% Lipofectamine, 0.025 wt-% pCEPluc plasmid). Animals were harvested after 24 hours by means of 8 mm skin punch. Gene expression was measured by assaying for luciferase activity in minced and permeabilized cell extracts, using a luminometer and data were normalized per protein content. The commercial luciferase assay kit (Sigma) was used. In another set of experiments, empty nanoparticles were used as another negative control with values of RLU/protein close to the negative control.

Results are presented in FIG. 5. The values presented as a bar height represent the average (n=number of sites)+/−SD. They clearly show that the formulated plasmid can achieve quite efficient gene transfection, many times over the baseline (controls) (about 400 times over the negative control). Similar results were obtained for polyanionic solution containing 0.025 wt-% pCEPluc and 0.005 wt-% SA-HV and polycationic solution containing 0.05 wt-% Tetronic 904 and 0.005 wt-% CaCl₂ in water. Some other detergents of the Pluronic and Tetronic series (BASF) worked equally well.

### EXAMPLE 23

Nanoparticles with Entrapped Growth Factor Protein

Nanoparticles were prepared the following way. The core solution contained 0.05% HV and 0.05% CS whereas the shell solution consisted of 0.05% SP, 0.15% PMCG, 0.05% calcium chloride and 1% F-68. The core solution also contained 0.00005-0.0005 wt % of fibroblast growth factor beta (FGFb)(Sigma). The ratio of core to shell solution was 2/20 (ml/ml). Once the nanoparticles were prepared, the in vitro release rates were evaluated in different buffers and two different temperatures (20° C. and 37° C.). At different time intervals, the solution (buffer) containing the released protein was removed for quantitation and replaced with new buffer. Daily replacement was used. bFGF release was measured via ELISA kit (Quantikine; R&D Systems, Minneapolis, Minn.), following the manufacturer's protocol. The biological activity of released bFGF was assayed via a proliferation assay (CellTiter 96 AQᵤₑₒᵤₛ One Solution Cell Proliferation Assay), based on formazan formation using MTS tetrazolium compound (TB 245, Promega, Madison, Wis.). NIH 3T3 fibroblasts, plated at a density of 2000 cells per well (96 well plate) were serum-starved for 16 hours in Optimem medium prior the 3-5 day assay.

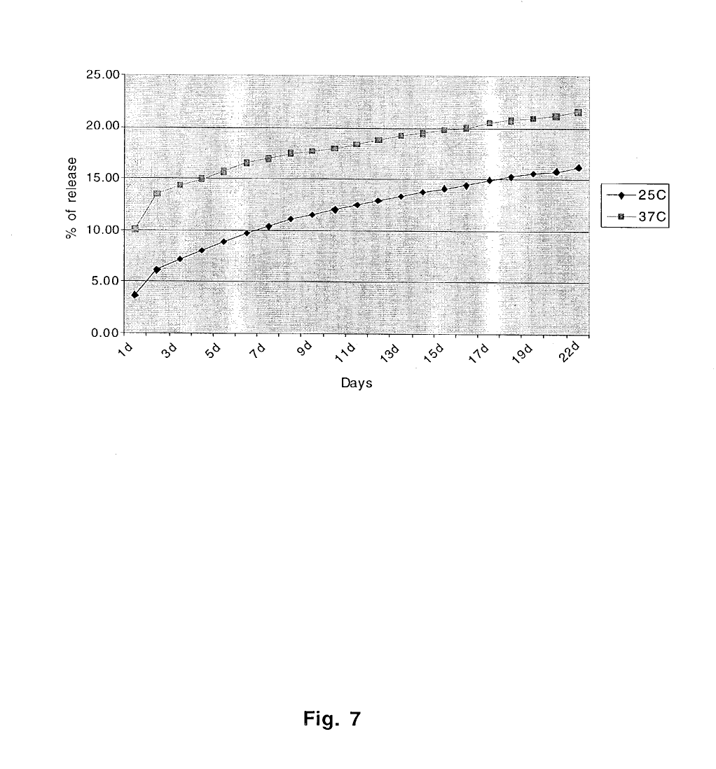

For detailed bFGF release studies, the protein was labeled (¹²⁵I) with help of IODO-BEADS iodination reagent (Pierce, Rockford, Ill.). An aqueous solution of bFGF at 6.6 mg/ml (100 μl) was added into 200 pi of reaction buffer (20 mM sodium phosphate pH 7.5, 150 mM NaCl) containing one polystyrene bead with immobilized iodination reagent and 2.5 μl of Na ¹²⁵I solution (13674 MBq/ml in 0.1 N NaOH, NEN, Perkin Elmer, Shelton, Conn.). After 5 minutes at room temperature the reaction was stopped by removing the solution from the reaction vessel and the labeled protein was passed trough a PD-10 column (Amersham, Arlington Heights, Ill.) in order to remove uncoupled, free ¹²⁵I molecules. The labeled protein was eluted by adding the reaction buffer containing 0.1% BSA to the column. Aliquots (5 μl) of all fractions (500 μl) were counted for a gamma radiation with a gamma counter to identify the labeled protein. The release of bFGF was between 17 to 32% over a 3-week period (FIG. 7). Although FGFb is a basic (cationic) protein, the concentrations added to a polyanionic core solution are so low that no insoluble complex between the FGFb and core polymers is formed. As many other basic growth factor proteins are used in vivo in low concentrations, it is contemplated that these proteins could be entrapped within the polyanionic core of the nanoparticle.

### EXAMPLE 24

Nanoparticles have Increased Entrapment Efficiency and Stability in the Presence of Salt and Sera

The nanoparticles are stable in presence of salts and sera. Even when the nanoparticles are made in presence of salts, the stability of the polyelectrolyte complex is extremely high. In addition, the entrapment efficiency is increased many times. Salts are known to destabilize the polyelectrolyte complexes and this often leads to their dissolution and ineffectiveness as a means of delivering a drug or other molecule, particularly intravenously. The nanoparticles may be prepared by the method described in Example 1. Alternatively, an increased amount of a monovalent or bivalent salt, e.g. sodium chloride, calcium chloride, or sodium sulfate may be added to the anionic solution prior to forming the polyelectrolyte complexes as in Example 1.

The stability of two nanoparticles prepared in the presence of sodium chloride and their entrapment efficiency were monitored. ACCSP nanoparticles were generated using a droplet-forming polyanionic solution composed of core solution containing 0.05% sodium alginate (Kelco), 0.05% cellulose sulfate (Janssen Chimica) and corona solution containing 0.05% calcium chloride (Sigma) and 0.075% poly(methylene-co-guanidine) chloride (Scientific Polymer Products), 0.05% Spermine hydrochloride and 1% F-68 (Pluronic). 2 milliliters of core solution is collected in 50 milliliters of corona solution. In addition, the core solution contained 0 wt % to about 2.0 wt % sodium chloride.

Table VI shows the entrapment efficiency of ACCSP, a quaternary complex, prepared in presence of sodium chloride solutions ranging from about 0 to 3 wt %. Stability was tested over a period of several months in 0.9% NaCl solution and sera. The nanoparticles were measured daily by a Malvern ZetaSizer instrument over a period of several weeks. For stability in sera, nanoparticles were resuspended in mice serum and size monitored over the required period of time. While the size in 0.9 wt % sodium chloride increased slowly over time, i.e., doubled in 2 weeks, the size of the nanoparticles prepared in the presence of a core sodium chloride concentration of about 1-3 wt % remained stable for long period of time. The size of nanoparticles remained the same as initially measured, i.e., 220 nm+/−25 nm. In both tests in 0.9 wt % NaCl and in serum, the amount of precipitate did not change with time, that is the amount of mass produced was stable.

TABLE VI
Stability of ACCSP prepared in presence of sodium chloride and effect
on entrapment efficiency

| Amount of NaCl in | Entrapment Efficiency | |
|---|---|---|
| Core solution (%) | % | Stability |
| 0 | 4.1 | extremely high |
| 1.00 | 13.1 | extremely high |
| 1.50 | 19.5 | extremely high |
| 2.00 | 28.3 | extremely high |
| 2.50 | 33.4 | extremely high |
| 3.00 | 38.5 | extremely high |

Similar data were obtained for calcium chloride and sodium sulfate solutions in the range of 0-3 wt %. Similar results as in Table VI were obtained for protein-loaded nanoparticles (data not shown). Intravenous application of nanoparticles into mice (tail vein) also corroborates the stability of these nanoparticles and their nonaggregation in vivo.

The following references are cited herein:

1. Desay, P. B., Microencapsulation of drugs by pan and air suspension technique. Crit. Rev. Therapeut. Drug Carrier Syst., 5: 99-139 (1988)

2. Berthold, et al., Preparation and characterization of chitosan microspheres as drug carrier for prednisolone sodium phosphate as model anti-inflammatory drugs. J. Controlled Release 39: 17-25 (1996)

3. Watts, P. J., Davies, H. C., Melia, C. D. Microencapsulation using emulsification/solvent evaporation: An overview of techniques and applications. Crit. Rev. Therapeut. Drug Carrier Syst. 7: 235-159 (1990)

4. Cowsar, et al., Poly(lactide-co-glycolide) microcapsules for controlled release of steroids. Methods Enzymol. 112: 101-116 (1985)

5. Genta, et al., Spray-drying for the preparation of chitosan microspheres. Proc. Int. Symp. Controlled Release Mater. 21: 616-617 (1994)

6. Cohen, et al., Controlled delivery systems for proteins based on poly(lactic/glycolic acid) microspheres. Pharm. Res. 8: 713-720 (1991)

7. Al-Khouri-Fallouh, et al., Development of new process for the manufacture of polyisobutylcyanoacrylate nanoparticles. Int. J Pharm. 28: 125-132 (1986)

8. Couvreur, et al., Biodegradable submicroscopic particles containing a biologically active substance and compositions containing them, U.S. Pat. No. 4,329,332 (1982)

9. Cruz, et al., Interaction between polyalkylcyanoacrylate nanoparticles and peritoneal macrophages: MTT metabolism, NBT reduction, and NO production. Pharm. Res. 14: 73-79 (1997)

10. Chang, T. M. Hybrid artificial cells: Microencapsulation of living cells. ASAIO Journal 38: 128-130 (1992)

11. Dautzenberg et al., Polyelectrolytes. Formation, Characterization and Applications, Hanser Publishers, Munich, 1994

12. Izumrudov, et al., Controllable stability of DNA-containing polyelectrolyte complexes in water-salt solutions, Biopolymers 52: 94-108, 1999

13. Liu, Yao and Lui, Formation of a DNA/N-dodecylated chitosan complex and salt-induced gene delivery, Journal of Applied Polymer Science 82: 3391-3395 (2001)

14. Dragan and Cristea, Polyelectrolyte complexes IV. Interelectrolyte complexes between some polycations with N,N-dimethyl-2-hydroxypropyleneammonium chloride units and poly(sodium styrenesulfonate) in dilute aqueous solution, Polymer 43: 55-62 (2002).

15. Zintchenko, et al., Polyelectrolyte complex formation with double hydrophilic block polyelectrolytes: Effects of the amount and length of the neutral block. Langmuir 18: 1386-1393 (2002).

16. Mao et al. Chitosan-DNA nanoparticles as gene carriers: synthesis, characterization and transfection efficiency. Journal of Controlled Release 70: 399-421 (2001).

17. Sternberg et al., New structures in complex formation between DNA and cationic liposomes visualized by freeze-fracture electron microscopy. FEBS Letters 356: 361-366 (1994).

18. Milson, R. W. and Bloomfield, V. A. Counterion-induced condensation of DNA. A light-scattering study. Biochemistry 18: 2192-2196 (1979).

19. Altmann, K. G. Effect of cationization on anti-hapten antibody response in sheep and mice. Immunol. Cell Biology 71: 517-525 (1993)

20. Emanuele et al., Polyoxypropylene/polyoxyethylene copolymers with improved biological activity, U.S. Pat. No. 5,567,859 (1996)

21. Hunter, R. J. Methods and vaccines comprising surface-active copolymers, U.S. Pat. No. 5,534,372 (1996).

22. Challacombe, et al., Enhanced secretory IgA and systemic IgG antibody response after oral immunization with biodegradable microcapsules containing antigen. Immunology 76: 164-168 (1992)

23. Ichimura, K., Watanabe, S. Preparation and characterization of photo-crosslinkable poly(vinyl alcohol). J. Polymer Science, Polymer Chemistry Edition 20: 1419-1432 (1982)

24. Schiller, et al., Characterization of the Pseudomonas aeruginosa alginate lyase gene (algL): Cloning, sequencing, and expression in Escherichia coli. J. Bacteriology 175: 4780-4789 (1993)

25. Cox, J. C., Coulter, A. R., Adjuvants—a classification and review of their modes of action, Vaccine 15:248-256 (1997)

Any patents or publications mentioned in this specification are indicative of the levels of those skilled in the art to which the invention pertains. These patents and publications are herein incorporated by reference to the same extent as if each individual publication was specifically and individually incorporated by reference.

One skilled in the art will readily appreciate that the present invention is well adapted to carry out the objects and obtain the ends and advantages mentioned, as well as those inherent therein. The present examples along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of making a nanoparticle that is stable in a physiological environment for at least a day, said nanoparticle comprising polyanionic polymers and polycations in a complex useful for drug delivery, said method comprising the step of:
capturing droplets comprising said polyanionic polymers in a solution comprising said polycations; or, alternatively,
capturing droplets comprising said polycations in a solution comprising said polyanionic polymers.
wherein the complex structure forming the nanoparticle made thereby increases the stability thereof in a physiological environment

2. The method of claim 1, wherein said physiological environment is a physiological media or sera.

3. The method of claim 1, wherein said polyanionic droplets or said polycationic droplets are in a mist, are uniformly sized in a stream or are in a solution.

4. The method of claim 1, wherein said polyanions and polycations are mixed together in the ratio of from about 1:1 to about 1:4.

5. The method of claim 1, wherein each of said polyanions and each of said polycations are independently present in concentrations of about 0.01 wt-% to about 2.0 wt-%.

6. The method of claim 1, wherein the nanoparticles have a polyanionic core and polyanionic/polycationic complex corona with an excess positive charge on the particle periphery.

7. The method of claim 6, wherein the nanoparticle comprises a ternary or quaternary complex.

8. The method of claim 6, wherein said polyanionic core further comprises an anionic protein, antigen or a nucleic acid incorporated as an integral component of the polyanionic droplets into the polycationic solution to form the nanoparticle.

9. The method of claim 8, wherein said nucleic acid is plasmid DNA or an antisense RNA oligonucleotide

10. The method of claim 6, wherein said polyanionic core further comprises a monovalent or bivalent salt.

11. The method of claim 10, wherein said salt is sodium chloride, calcium chloride or sodium sulfate.

12. The method of claim 11, wherein salt is present in a concentration up to about 3 wt %.

13. The method of claim 1, wherein said polyanionic polymers or said polycationic polymers comprise corona polymers of said nanoparticle, further comprising a charged polymeric surface modifier of the same charge as the corona polymers, wherein said corona polymers and said charged surface modifier are incorporated in one step as an integral component of the nanoparticle

14. The method of claim 1, wherein said polyanionic polymers or said polycationic polymers further comprise a nonionic polymeric surface modifier, wherein said nonionic surface modifier is incorporated into the nanoparticle as a steric stabilizer.

15. The method of claim 1, wherein the nanoparticles have a polycationic core and a polycationic/polyanionic complex corona with the excess of negative charge on the particle periphery.

16. A method of increasing the entrapment efficiency of a nanoparticle comprising the steps:
capturing droplets comprising polyanionic polymers and a salt in a solution comprising polycations, said polyanionic polymers and said polycations sufficient in number to form a ternary or quaternary polymeric nanoparticle structure; and
forming said nanoparticle structure comprising a polyanionic/salt core and a polycationic/polyanionic complex corona with an excess positive charge on the nanoparticle periphery wherein the structure of the nanoparticle increases the stability thereof in a physiological environment thereby increasing entrapment efficiency.

17. The method of claim 16, wherein said polyanionic/salt droplets further comprise a anionic protein, an anionic antigen or a nucleic acid incorporated as an integral component of the polyanionic/salt core upon formation of the nanoparticle.

18. The method of claim 17, wherein said nucleic acid is plasmid DNA or an antisense RNA oligonucleotide

19. The method of claim 16, wherein said salt is a monovalent salt or a bivalent salt.

20. The method of claim 19, wherein said salt is sodium chloride, calcium chloride or sodium sulfate.

22. The method of claim 20, wherein salt is present in a concentration up to about 3 wt %.

23. The method of claim 16, wherein said physiological environment is a physiological media or sera.

24. The method of claim 16, wherein said polyanionic/salt droplets are in a mist, are uniformly sized in a stream or are in a solution.

25. The method of claim 16, wherein said polyanions and polycations are mixed together in the ratio of from about 1:1 to about 1:4.

26. The method of claim 16, wherein each of said polyanions and each of said polycations are present in concentrations of about 0.01 wt-% to about 2.0 wt-%.

27. A nanoparticle comprising:
a core comprising polyanionic polymers and a salt; and
a corona surrounding said core, said corona comprising a polycationic/polyanionic complex with an excess positive charge on the nanoparticle periphery, wherein said polyanionic polymers and said polycations are sufficient in number to form a ternary or quaternary polymeric nanoparticle structure.

28. The nanoparticle of claim 27, wherein said polyanionic/salt core further comprises an anionic protein, a cationic protein in an amount that does not insolubly complex with a polyanionic polymer in said core, an antigen or a nucleic acid.

29. The nanoparticle of claim 28, wherein said nucleic acid is plasmid DNA or an antisense RNA oligonucleotide

30. The nanoparticle of claim 27, wherein said salt is a monovalent or bivalent salt.

31. The nanoparticle of claim 30, wherein said salt is sodium chloride, calcium chloride or sodium sulfate.

32. The nanoparticle of claim 31, wherein salt is present in a concentration up to about 3 wt %.

33. A vaccine comprising the nanoparticle and the anionic antigen contained therein produced by the method of claim 8.

34. A method of treating an animal to elicit an immune response comprising the step of:
administering the vaccine of claim 33 to the animal, said vaccine containing an effective amount of said anionic antigen to elicit the immune response.

35. The method of claim 34, wherein said vaccine is administered orally or intravenously.

36. The method of claim 34, wherein the nanoparticles are administered orally to the animal, wherein the nanoparticles are taken up by M-cells in Peyer's patches of the epithelial lining of the upper intestinal tract of the animal resulting in an increase in secretory and systemic antibodies in blood.

37. A method of delivering a protein, an antigen or a nucleic acid to an animal to treat the animal comprising the step of:
administering the nanoparticle of claim 28 to the animal, said nanoparticle containing an effective amount of said anionic antigen or said nucleic acid to effect treatment of the animal.

38. The method of claim 37, wherein said nucleic acid is plasmid DNA or an antisense RNA oligonucleotide.

39. The method of claim 37, wherein the nanoparticle is administered orally or intravenously.

40. The method of claim 37, wherein said nanoparticle contains an amount of anionic antigen effective to illicit an immune response in the animal.

41. A vaccine comprising the nanoparticle and anionic antigen contained therein of claim 28.

42. A method of treating an animal to elicit an immune response comprising the step of:
administering the vaccine of claim 41 to the animal, said vaccine containing an effective amount of said anionic antigen to elicit the immune response.

43. The method of claim 42, wherein said vaccine is administered orally or intravenously.

44. A method of processing reactor content to remove unwanted residual reactants comprising the steps of:
sedimenting or centrifuging said reactor content;
collecting microparticles or collecting the nanoparticles of claim 1 generated in a stirred reactor;
rinsing said particles in an excess of water, buffer or cryopreservation solution;
separating said suspension by said sedimentation or centrifugation step;
repeating said rinsing and separation steps; and
reducing volume of the said suspension to about 1/100th of the initial volume.

45. A method for the chemical stabilization of the washed and isolated particles of claim 44, comprising the steps of:
reacting the particles with a crosslinking agent;
rinsing said particles in an excess of water, buffer or a cryopreservation solution;
separating the particles by sedimentation or centrifugation;
repeating the rinsing and separation steps; and reducing volume of the suspension.

46. The method of claim 45, wherein said crosslinking agent is selected from the group consisting of dextran polyaldehyde, a photocrosslinking polymer and a γ-glutamyl transferase enzyme.

47. A method of cryoprotecting the washed particles of claim 45, comprising the steps of:
suspending the particles in a cryoprotective solution to form a suspension; and
lyophilizing the suspension.

48. The method of claim 47, wherein said cryoprotective solution is selected from the group consisting of glycerol, trehalose, sucrose, PEG, PPG, PVP, block polymers of polyoxyethylene and polyoxypropylene, block polymers of polyethylene oxide and polyvinylacohol, polymeric 2-methacryloxyethylphosphorylcholine, and water soluble derivatized celluloses.

49. The method of claim 48, wherein said cryoprotective solution is in a concentration of 1 wt-% to 10 wt-%.

50. A method of adjustment of biodegradability of polymeric mixtures, comprising the steps of:
contacting an enzyme to a polysaccharide;
degrading said polysaccharide at physiological conditions in vivo to degradation products.

51. The method of claim 50, wherein said enzyme is selected from the group consisting of alginate-lyase (alginase) and carrageenase for polymer matrices containing alginate or carrageenans.

52. A method of introducing an adjuvant to potentiate an immunogenic effect, comprising the steps of:
administering said adjuvant as part of a droplet-forming polymeric mixture.

53. The method of claim 52, wherein said adjuvant is aluminum salt enabling to gel certain polysaccharides.

54. The method of claim 52, wherein said adjuvant is CMC, CS and HV alginate as droplet forming anionic polymers or aluminum sulfate, calcium chloride and a polycationic polymers as a corona forming mixture.
